(19) United States
(12) Patent Application Publication
(10) Pub. No.: US 2019/0192103 A1
(43) Pub. Date: Jun. 27, 2019

(54) METHOD FOR AUTOMATICALLY GENERATING A VOLUME MODEL OF CORRECTION DATA FOR AN X-RAY BASED MEDICAL IMAGING DEVICE

(71) Applicant: Siemens Healthcare GmbH, Erlangen (DE)

(72) Inventors: André Ritter, Neunkirchen am Brand (DE); Christian Hofmann, Erlangen (DE); Pavlo Dyban, Berlin (DE); Jens-Christoph Georgi, Oberasbach (DE); Kai Schubert, Heidelberg (DE); Dieter Oetzel, Oftersheim (DE); Eric Tonndorf-Martini, Heidelberg (DE)

(21) Appl. No.: 16/225,933

(22) Filed: Dec. 19, 2018

(30) Foreign Application Priority Data
Dec. 21, 2017 (DE) 10 2017 223 604.3

(51) Int. Cl. A61B 6/00; A61B 6/12; G06T 7/13; G06T 7/11; G06T 15/08

(52) U.S. Cl. CPC A61B 6/5282; A61B 6/12; G06T 7/13; G06T 2207/10116; G06T 15/08; G06T 2207/30052; G06T 2207/10081; G06T 7/11

(57) ABSTRACT

Method and systems are provided for automatically generating a volume model of correction data for an X-ray based medical imaging device. A plurality of X-ray images is recorded of a body region of a patient to be examined from different positions in each case. The plurality of X-ray images is used to generate a first volume model of the body region. Image artifacts are corrected in the first volume model using the plurality of X-ray images and thus a corrected volume model is generated. The corrected volume model is used to determine a contour of an artifact volume affected by image artifacts in the first volume model and the contour of the artifact volume is defined as a volume model of correction data. The volume model of correction data is stored on a data medium and/or output via an interface.

### CROSS REFERENCE TO RELATED APPLICATIONS

This application claims the benefit of DE 10 2017 223 604.3 filed on Dec. 21, 2017, which is hereby incorporated by reference in its entirety.

### FIELD

Embodiments relate to a method for automatically generating a volume model of correction data for an X-ray based medical imaging device.

### BACKGROUND

When planning radiotherapy, as used, for example, to control a tumor, it is usual to ascertain physical parameters of the irradiation, for example angle of incidence, radiation dose and profile, based on medical image data, that is generated by computed tomography (CT). For the planning, individual regions that each correspond to different tissue structures and also include the tumor tissue are identified in the image data. Knowledge of the spatial distribution of the different tissue structures will provide the optimal dose distribution to be calculated, e.g. the maximum possible irradiation dose in the tumor tissue in conjunction with the lowest possible radiation dose in the other tissue structures. It is also possible to make special distinctions regarding the latter depending upon the radiation intensity.

The quality of reproduction of the image data provided is of crucial importance if the planning is to meet the criteria for the dose distribution. However, if the body tissue to be mapped by the CT scanner for radiotherapy planning contains a foreign body that absorbs X-rays to a significantly different degree than the surrounding body tissue, the image data output by the CT scanner might contain artifacts that do not correspond to the real situation in the mapped body tissue. Such a foreign body may, for example, take the form of medical implants, such as, for example, bone, joint or cochlea implants or even dental fillings, cardiac pacemakers, aneurysm coils or clips. Such foreign bodies may include a significantly higher density than the surrounding body tissue so that, when recording an individual X-ray image, it is no longer possible to make meaningful statements relating to the region that is shaded by the foreign body from the X-ray source of the CT scanner due to the much higher absorption by the foreign body. The reconstruction of the volume model of the body region to be examined from a plurality of such X-ray images in which a large region no longer supplies any useful absorption information results in the occurrence of regions in the volume model corresponding to an apparently high degree of absorption not only at the site of the actual foreign body. The faulty absorption information may also result in the occurrence of zones with apparently higher or even lower absorption or apparently inhomogeneous tissue in the environment of the foreign body in the volume model.

To provide effective radiotherapy planning based on such artifact-laden image data, there are now user environments in which the boundaries of different volumes may be plotted manually or semi-automatically by confirming a suggestion. The regions plotted in this way are assigned properties intended to provide a specific dose calculation, such as, for example, manually overwriting the respective CT image data with specific HU values at the respective volume element (voxel). However, this is extremely complicated. In addition, correctly identifying the individual boundaries and interfaces in the artefact-laden image data requires a high level of experience. Herein, in the worst case, human errors may result in scenarios where highly sensitive tissue covered by an artifact is not detected correctly and hence receives an excessive dose of irradiation.

In addition, there are also possibilities, for example with metallic foreign bodies, for correcting artifacts in the image data to the greatest degree possible. However, when using the corrected image data, the quality of the radiotherapy planning is dependent on the quality of the correction. There is also a residual risk of critical body tissue being covered by the artifacts in the original image data so that the critical body tissue may no longer be identifiable as such in the corrected image data after correction of the artifacts. It is not least for this reason that radiotherapy planning based solely on artifact-corrected image data is often rejected.

### SUMMARY AND DESCRIPTION

The scope of the present disclosure is defined solely by the appended claims and is not affected to any degree by the statements within this summary. The present embodiments may obviate one or more of the drawbacks or limitations in the related art.

Embodiments include a method for generating correction data from medical, for example three-dimensional, image data that provides radiotherapy to be planned as optimally as possible, even in the presence of image artifacts. Embodiments further include a method for processing three-dimensional image data for calculating irradiation.

A method for automatically generating a volume model of correction data for an X-ray based medical imaging device is provided. A plurality of X-ray images is recorded of a body region of a patient to be examined from different positions in each case. The plurality of X-ray images is used to generate a first volume model of the body region. Image artifacts are corrected in the first volume model using the plurality of X-ray images and thus a corrected volume model is generated. The corrected volume model is used to determine a contour of an artifact volume affected by the first volume model image. The contour of the artifact volume is defined as a volume model of correction data and the volume model of correction data is stored on a data medium and/or output via an interface.

Automatic generation of the volume model of correction data may imply that all the method steps are performed on and by a computer. An X-ray based medical imaging device may be understood to imply a device that uses for imaging a modality based on the physical principle of X-rays and the absorption thereof by body tissue. For example, an X-ray based medical imaging device may include a CT scanner or a comparable modality in which three-dimensional image data is obtained by a reconstruction method by inverse transformation from a plurality of individual recordings.

A volume model may refer to a function dependent on one of three local arguments, where for the first volume model and for the corrected volume model, the specific function value at a specific point inside the volume is in the form of a scalar representing the degree of X-ray absorption at the point in question. The graphical depiction of the scalar values supplies three-dimensional image data of the body region depicted by the X-ray images. However, the function values for the volume model of correction data are of a binary nature and only relate to the distinction as to whether a specific point in three-dimensional position space lies within the contour of the artifact volume. The position space may be finely discretized. A lower limit of resolution may be the image resolution of the X-ray detector during the recording of the individual X-ray images. For example, it is possible to refer to volume elements (voxels). A volume element is the smallest unit of volume that may be resolved by the medical imaging device.

The plurality of X-ray images of the body region may be recorded in each case from different angular and/or axial positions of the X-ray source relative to the patient. The generation of the first volume model of the body region using the plurality of X-ray images may be performed using an inverse tomographic transform, such as, for example, that in the inverse Radon transform.

An image artifact in the first volume model may be image information that does not correspond to the tissue structures actually present in the body region but only occurs as a result of the reconstruction for generating the first volume model from the plurality of X-ray images. The image information of an image artifact for individual X-ray images is inconsistent. The first volume model may, for example, be corrected using empirical values and statistical methods. For example, the image values for individual volume elements may be corrected iteratively. First, corrected image values may be determined for a number of volume elements and then compatibility and consistency checked with the image values of other, as yet uncorrected volume elements and, if necessary, adjusted once again. The corrected volume model may as such be understood to imply the totality of all the items of image information for the individual volume elements that include the corrected image values at the corresponding places and, for volume elements for which the image values have not been corrected, retain the original image values of the first volume model.

An artifact volume in the first volume model may imply the totality of the volume elements with artifact-laden image values, e.g. that do not reflect the real tissue structures in the body region to be depicted. The contour of the artifact volume may imply a simply coherent area within the limit of resolution as set by the volume elements in the interior of which the artifact-laden volume elements are located. A plurality of simply coherent areas may each be defined as a contour. Only the location information for the individual volume elements forming the contour may be defined as the volume model of correction data and the volume model of correction data saved or output correspondingly.

The procedure described provides the original three-dimensional image data as represented in the first volume model to be used when planning radiotherapy. However, additional information is provided regarding the regions of this image data that might contain image artifacts. Special caution is required in the interpretation of this image data and in further processing in the form of the segmentation of individual tissue structures or the like. Vice versa, the volume model of correction data also provides that, outside the contour of the artifact volume, the image information supplied in the first volume model does not contain any significant image artifacts, but may be presumed to be a sufficiently accurate reflection of the corresponding tissue structures. This also greatly simplifies radiotherapy planning since now no manual or semi-automatic corrections are needed in these regions, saving time.

The contour of the artifact volume may be additionally determined using the plurality of X-ray images. Depending on the type of errors, that may result in artifacts on reconstruction, the X-ray images available before reconstruction to form the first volume model may nevertheless contain information, that although it does not permit independent, artifact-free reconstruction solely based on the X-ray images, may still be utilized for an additional check on the correction data determined using the corrected volume model, e.g. in the form of the contour of the artifact volume. Such information is, for example, found in residual absorption contrasts, that, as a result of the great differences in all the absorption values that occur, have no significance for back-projection, but may be used to check the plausibility of the contour of the artifact volume.

To generate the corrected volume model in the first volume model, the image artifacts corrected are caused by at least one foreign body. Herein, a foreign body may be understood to imply a structure within a body region, that is not formed by body tissue, e.g. a medical implant, but also jewelry or the like. Herein, due to its material composition, the foreign body is much more absorbent to X-rays than the surrounding body tissue. For such foreign bodies, due to the shading of the X-rays, a high number of image artifacts occur on three-dimensional reconstruction from the individual X-ray images.

A first contour of the foreign body may be ascertained in the first volume model using the corrected volume model, and also using the plurality of X-ray images and/or the artifact volume and for the first contour of the foreign body to be included in the volume model of correction data. This makes it possible, when planning radiotherapy, not only to identify the regions in which image artifacts might be present in the first volume model containing the uncorrected three-dimensional image data, but also to take account of the position and spatial extension of the foreign body responsible for the image artifacts. This makes it possible, for example, to avoid shading effects from the foreign body during radiotherapy that might have a detrimental effect on the dose distribution. The inclusion of the first contour in the volume model of the correction data may take place in a similar manner to that of the contour of the artifact volume.

A second contour of a homogeneous region of the foreign body is ascertained within the first contour of the foreign body, for example using the corrected volume model, and the second contour of the homogeneous region is included in the volume model of correction data. A homogeneous region entails a region in the foreign body made of a uniform material. Thus, additional information on an internal structure of the foreign body is provided. In the homogeneous region, the foreign body includes uniform absorption properties that may also be taken into account when planning radiotherapy. The inclusion of the second contour in the volume model of the correction data take place in a similar way to that of the contour of the artifact volume. The correction data in the volume model relating to the first contour and, if present, also the second contour, may be taken into account in the segmentation of image regions corresponding to tissue structures for planning radiotherapy.

The contour of a medical implant is ascertained as the first contour. This may be the case for foreign bodies that give rise to image artifacts in three-dimensional image data reconstructed from a plurality of X-ray images, especially since, unlike many types of jewelry, a medical implant, may not be removed from the body tissue for X-ray imaging.

In an embodiment, the first volume model and the corrected volume model are used to form a function of a correction depth. The contour of the artifact volume is determined by a comparison of the function of the correction depth with a prespecified limit value. The determination of a function of the correction depth first provides gradual statements to be made with respect to the correction applied and also the visualization thereof. Herein, the limit value may be specified in dependence on the function of the correction depth, and in dependence on their value range. If the limit value is exceeded, a binary value signaling the presence of an image artifact is set for a corresponding volume element. The totality of all such volume elements then forms the artifact volume and an area enclosing the artifact volume, and possibly also including volume elements for which the corresponding binary value, indicates the absence of image artifacts may then be accepted as the corresponding contour.

The function of the correction depth in each volume element, e.g. voxel-by-voxel, is formed from the absolute value of the difference between the value of the first volume model and the value of the corrected volume model in the volume element. This is implemented mathematically and provides accurate results due to the linearity in the amount of the difference.

In an embodiment, a method for automatically processing a volume model of medical image data for calculating irradiation is provided. For a body region of a patient, a first volume model and a volume model of correction data are generated by a method. In the first volume model, individual regions that each correspond to different tissue structures are segmented by a computer. The volume model of correction data for calculating irradiation is incorporated in the segmented regions. The circumstance is exploited that that the tissue structures mapped in the first volume model are to be segmented for informative radiotherapy planning in order, inter alia, to provide tissue with identical biological properties to be treated in the same way in the dose calculation. The inclusion of the volume information with respect to the first and possibly the second contour of a foreign body provides such a foreign body to be taken into account directly in the calculations of the dose distribution for a specific beam profile.

Embodiments further include a computer program product with program code for carrying out the above-described method for automatically generating a volume model of correction data for an X-ray based medical imaging device when the computer program is executed on a computer.

Embodiments also include an X-ray based medical imaging device including at least one X-ray source for generating an X-ray beam, an X-ray detector for recording X-ray images and a computing unit configured to carry out the above-described method for automatically generating a volume model of correction data. When used as prescribed, the X-ray based medical imaging device uses a plurality of X-ray images of a body region of a patient to be examined to generate a volume model of the body region. The X-ray based medical imaging device may be configured as a CT scanner. The advantage of a device configured in this way is that the volume model of correction data is generated in the same place that the unprocessed X-ray images are generated and are hence available without any loss of quality. In subsequent processing of the medical image data, following three-dimensional reconstruction from the plurality of X-ray images, the latter are often not available, or only available in compressed form, in order to reduce the required storage capacities.

### BRIEF DESCRIPTION OF THE FIGURES

FIG. 1 depicts an example cross-sectional view of a computed tomography scanner.

FIG. 2 depicts an example block flow diagram of a method for generating a volume model of correction data for the CT scanner in FIG. 1.

### DETAILED DESCRIPTION

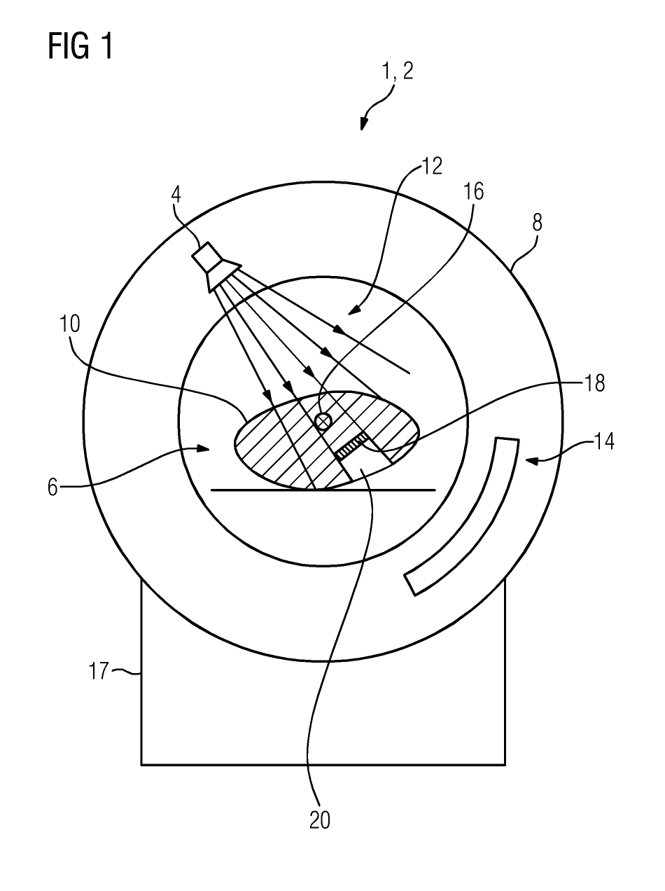

FIG. 1 depicts a schematic cross-sectional view of an X-ray based medical imaging device 1 that is configured as a CT scanner 2. In the CT scanner 2, an X-ray source 4 irradiates a body region 10 of a patient positioned in the interior 6 of the rotating ring 8 of the CT with X-rays 12. The portions of the X-rays 12 that are not absorbed by the body region 10 of the patient are measured on the opposite side relative to the interior 6 of the X-ray source 4 by an X-ray detector 14 and processed to form an individual X-ray image. For complete imaging, different X-ray images are recorded. For the individual recordings, both the X-ray source 4 and the X-ray detector 14 rotate around an axis 16 perpendicular to the image plane. There may be an axial displacement of the X-ray source 4 and X-ray detector 14 along the axis 16. Both the X-ray source 4 and the X-ray detector 14 perform the movement of discretized coverage of a cylinder surface. The individual X-ray images are then transferred to a retaining frame 17 where a three-dimensional volume model of the body region 10 is created by back projection.

If there is a foreign body 18 in the body region to be examined 10, that may, for example, be in the form of a medical implant, depending upon the angular position of the X-ray source 4 and the X-ray detector 14, the foreign body 18 shades parts of the X-rays 12 so that the X-rays 12 no longer provide accurate information on the tissue 20 that is shaded from the X-ray source 4. The totality of such shading effects in the X-ray images may result in image artifacts on the three-dimensional reconstruction that inter alia could significantly complicate radiotherapy planning, for example, for tumor control.

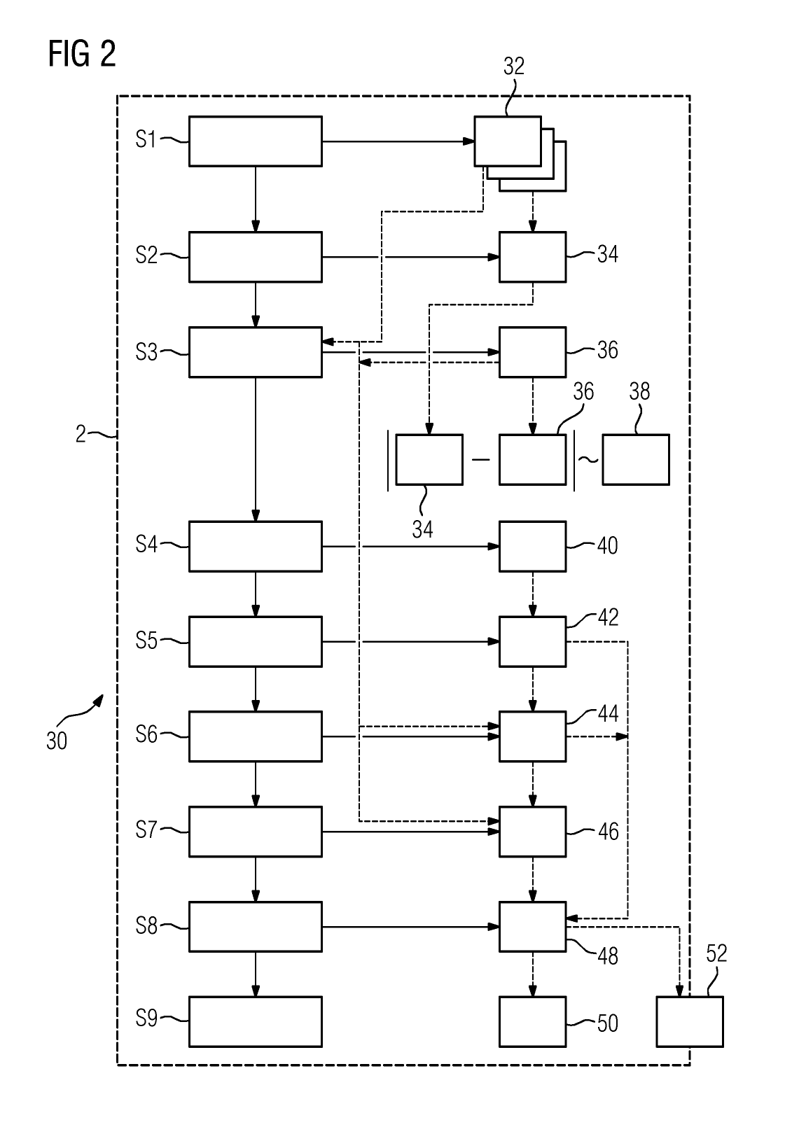

FIG. 2 depicts a schematic block flow diagram of a method 30 performed in the computed tomography scanner 2 depicted in FIG. 1. At act S1, a plurality of X-ray images 32 of the body region 10 of the patient to be examined are recorded from different angular and axial positions in each case. The X-ray images 32 are in each case transferred from the rotating ring 8 to the retaining frame 17, where at act S2, a three-dimensional, first volume model 34 of the body region 10 is generated by inverse transformation. The first volume model may now by appraised by a physician or a medical physician. If, as a result of a foreign body 18 in the body region to be depicted 10, the first volume model 34 contains image artifacts, the image artifacts are corrected in a correction act S3 using information in the X-ray images 32. The result of this correction is a corrected volume model 36. At act S4, the difference between the image values of the first volume model 34 and the corrected volume model 36 is formed for each individual volume element, e.g. voxel-by-voxel, and the absolute value obtained. This is compared to a prespecified limit value 38 thus providing, in the event of the limit value being exceeded, the conclusion to be drawn that there is an image artifact 40 in the present volume element.

At act S5, a contour 42 is determined, that as a coherent area encloses the totality of all the volume elements affected by image artifacts 40.

At act S6, the corrected volume model 36 and the X-ray images 32 and the contour 42 enclosing the image artifacts 40 are used to ascertain a first contour 44 of the foreign body 18 in the first volume model 34. The first contour 44 in the first volume model 34 encloses the volume elements corresponding to the foreign body 18 in the body region. At act S7, the information obtained so far is used to determine a second contour 46 of a region within the foreign body 18 that is homogeneous with respect to its material composition within the first contour of the foreign body 18. This may, for example, in a medical implant that is formed from both metal and ceramic components, be one of the two components. At act S8, the contour 42 enclosing the image artifacts 40, the first contour 44 of the foreign body 18, and the second contour 46 representing a homogeneous region in the foreign body 18 are defined as correction data 48 and then, at act S9, both stored on a data medium 50 and output via an interface 52 of the CT scanner 2. The outputting via the interface 52 may take place on a separate computer on which the actual radiotherapy planning is to be performed.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for automatically generating a volume model of correction data for an X-ray based medical imaging device, the method comprising:
recording a plurality of X-ray images for a body region of a patient to be examined from different positions;
generating a first volume model of the body region using the plurality of X-ray images;
generating a corrected volume model, generating the corrected volume model comprising correcting image artifacts in the first volume model using the plurality of X-ray images;
determining a contour of an artifact volume affected by image artifacts in the first volume model using the corrected volume model, wherein the contour of the artifact volume is defined as a volume model of correction data; and
storing the volume model of correction data on a data medium, outputting the volume model of correction data via an interface, or storing the volume model of correction data on the data medium and outputting the volume model of correction data via the interface.

2. The method of claim 1, wherein the contour of the artifact volume is also determined using the plurality of X-ray images.

3. The method of claim 1, wherein correcting the image artifacts in the first volume model comprises correcting image artifacts caused by at least one foreign body in the first volume model.

4. The method of claim 3, further comprising determining a first contour of the foreign body using the corrected volume model,
wherein the first contour of the foreign body is included in the volume model of correction data.

5. The method of claim 4, further comprising determining a second contour within the first contour of the foreign body, the second contour being of a homogenous region of the foreign body,
wherein the second contour of the homogeneous region is included in the volume model of correction data.

6. The method of claim 4, wherein a contour of a medical implant is determined as the first contour.

7. The method of claim 1, further comprising forming a function of a correction depth, forming the function of the correction depth comprising using the first volume model and the corrected volume model,
wherein determining the contour of the artifact volume comprises comparing the function of the correction depth with a prespecified limit value.

8. The method of claim 7, wherein the function of the correction depth in each volume element is formed from an absolute value of a difference between a value of the first volume model and a value of the corrected volume model in the volume element.

9. A method for automatically processing a volume model of medical image data for calculating irradiation, the method comprising:
generating, for a body region of a patient, a first volume model and a volume model of correction data, the generating of the first volume model and the volume model of correction data comprising:
recording a plurality of X-ray images for the body region of the patient to be examined from different positions;
generating the first volume model of the body region using the plurality of X-ray images;
generating a corrected volume model, generating the corrected volume model comprising correcting image artifacts in the first volume model using the plurality of X-ray images;
determining a contour of an artifact volume affected by image artifacts in the first volume model using the corrected volume model, wherein the contour of the artifact volume is defined as a volume model of correction data; and
storing the volume model of correction data on a data medium, outputting the volume model of correction data via an interface, or storing the volume model of correction data on the data medium and outputting the volume model of correction data via the interface;
segmenting, in the first volume model, individual regions that each correspond to different tissue structures; and
incorporating the volume model of correction data for calculating irradiation is incorporated in the segmented regions.

10. A non-transitory computer-readable storage medium that stores instructions executable by at least one processor to automatically generate a volume model of correction data for an X-ray based medical imaging device, the instructions comprising:
recording a plurality of X-ray images for a body region of a patient to be examined from different positions;
generating a first volume model of the body region using the plurality of X-ray images;
generating a corrected volume model, generating the corrected volume model comprising correcting image artifacts in the first volume model using the plurality of X-ray images;
determining a contour of an artifact volume affected by image artifacts in the first volume model using the corrected volume model, wherein the contour of the artifact volume is defined as a volume model of correction data; and
storing the volume model of correction data on a data medium, outputting the volume model of correction data via an interface, or storing the volume model of correction data on the data medium and outputting the volume model of correction data via the interface.

11. An X-ray based medical imaging device comprising:
at least one X-ray source configured to generate an X-ray beam;
an X-ray detector configured to record a plurality of X-ray images for a body region of a patient; and
a computer configured to generate, using the plurality of X-ray images, a first volume model of the body region, generate a corrected volume model, the generation of the corrected volume model comprising correction of image artifacts in the first volume model using the plurality of X-ray images, determine, using the corrected volume model, a contour of an artifact volume affected by image artifacts in the first volume model, and store the volume model of correction data on a data medium, output the volume model of correction data via an interface, or store the volume model of correction data on the data medium and output the volume model of correction data via the interface.

12. The X-ray based medical imaging device of claim 11, wherein the contour of the artifact volume is additionally determined using the plurality of X-ray images.

13. The X-ray based medical imaging device of claim 11, wherein the computing unit is configured to correct image artifacts caused by at least one foreign body.

14. The X-ray based medical imaging device of claim 13, wherein the corrected volume model is used to determine a first contour of the foreign body, and the first contour of the foreign body is included in the volume model of correction data.

15. The X-ray based medical imaging device of claim 14, wherein a second contour of a homogeneous region of the foreign body is determined within the first contour of the foreign body, and the second contour of the homogeneous region is included in the volume model of correction data.

16. The X-ray based medical imaging device of claim 14, wherein the contour of a medical implant is determined as the first contour.

17. The X-ray based medical imaging device of claim 11, wherein the first volume model and the corrected volume model are used to form a function of a correction depth, and
wherein the contour of the artifact volume is determined by a comparison of the function of the correction depth with a prespecified limit value.

18. The X-ray based medical imaging device of claim 17, wherein the function of the correction depth in each volume element is formed from an absolute value of a difference between a value of the first volume model and a value of the corrected volume model in the volume element.

19. The X-ray based medical imaging device of claim 11, wherein the computing unit is further configured to segment, in the first volume model, individual regions that each correspond to different tissue structures,
wherein the volume model of correction data is incorporated in the segmented regions.
